(19) United States
(12) Patent Application Publication
(10) Pub. No.: US 2009/0176303 A1
(43) Pub. Date: Jul. 9, 2009

(54) Method and apparatus for propagating benthic marine invertebrates

(75) Inventors: Ami Schlesinger, D.N. Menashe (IL); Esther Kramarsky-Winter, Rehovot (IL); Yossi Loya, Raanana (IL)

Correspondence Address: THE NATH LAW GROUP, 112 South West Street, Alexandria, VA, 22314, US

(73) Assignee: Ramot at Tel-Aviv University Ltd, Tel Aviv (IL)

(21) Appl. No.: 12/320,946

(22) Filed: Feb. 9, 2009

Related U.S. Application Data
(63) Continuation-in-part of application No. PCT/IL2007/000908, filed on Jul. 18, 2007.

(30) Foreign Application Priority Data
Aug. 10, 2006 (IL) 177409

(51) Int. Cl. C12N 5/06; C12M 3/00

(52) U.S. Cl. 435/325; 435/293.1

(57) ABSTRACT

An apparatus for propagating benthic marine invertebrates in water. The apparatus comprises (a) a culture cell, (b) a sedimentation chamber in fluid connection with the culture cell, and (c) a pump in fluid connection with the culture cell and with the sedimentation chamber capable of pumping water from the sedimentation chamber to the culture cell. Also disclosed is a method for propagating benthic marine invertebrates comprising culturing the invertebrates in a water environment in the apparatus.

This is a Continuation-In-Part of International PCT Application No. PCT/IL2007/000908 filed Jul. 18, 2007 and claims priority from Israeli Patent Application no. 177409 filed Aug. 10, 2006, the contents of which are hereby incorporated by reference in their entirety.

### FIELD OF THE INVENTION

The present invention relates to a method and apparatus for propagating benthic marine invertebrates.

### BACKGROUND OF THE INVENTION

Molluscs are the second largest phylum in the animal kingdom and comprise a group of invertebrate animals, most of which have a three parted soft body: head, central mass and foot. The majority of molluscs are marine animals. The Phylum Mollusca includes the classes Gastropoda, Scaphopoda, Bivalvia and Cephalopoda. Representative examples of Gastropoda (“stomach-footed”) include snails, garden slugs and sea slugs. Naked-gill sea slugs are classified in the order Nudibranchia, and Aeolids in the suborder Aeolidacea.

Cnidarians are venomous marine invertebrates. The stinging apparatus of cnidarians (called a nematocyst) is a sub-cellular structure loaded with the venom it injects. Various types of nematocysts exist (there are 24 distinct morphological types), including: (1) poison containing cells; (2) cells containing harpoon-shaped barbs; and (3) cells containing sticky secretions or entangling coils.

The nematocyst consists of a capsule containing a highly folded reversible tubule. Discharge of the nematocyst is driven by the capsule's internal hydrostatic pressure of 150 atm, which causes eversion of the tubule with an acceleration of up to ca. 400,000 m·s⁻², making this event one of the fastest biotic mechanisms known to date.

The primary function of the penetrating type of nematocyst is the rapid delivery of complex mixtures of bioactive compounds, i.e., venoms, which can cause a variety of cytotoxic, neurotoxic, hemolytic, cardiotoxic, dermatonecrotic, immunogenic, and inflammatory effects. Cnidarian venoms may include vasoactive and neuroactive compounds, certain amino acids and small peptides, and proteins. Due to these pharmacological properties, cnidarian venoms are potentially valuable tools for biomedical research and drug development.

Cnidarian nematocysts are of added biotechnological interest, as an estimated 150 million people worldwide are exposed annually to jellyfish stings preparations of nematocysts may be useful in the development of repellents that prevent these stings. The cnidarian nematocysts may be also used as devices for delivering therapeutic, diagnostic, or cosmetic agents into a tissue.

The life cycles of both Molluscs and Cnidarians are complex, i.e., include a metamorphic stage, and Molluscs are often difficult to grow in captivity. Molluscs in general, and aeolid nudibranchs in particular, feed upon cnidarians. A partitioning of ingested, undischarged nematocysts from their prey occurs during digestion, and functional, unfired, nematocysts are sequestered in organs called cnidosacs. Large numbers of these nematocysts are discarded with the mollusc's faeces.

Cnidarian stinging cells are currently being exploited by the biomedical industry as platforms for drug delivery. For example, U.S. Pat. Nos. 6,613,344 and 6,923,976 describe compositions of matter comprising a therapeutic agent or a cosmetic agent and at least one stinging capsule derived from a stinging cell of a Cnidarian tentacle. To this end, stinging cells are isolated in an intact, dischargeable form from cnidarian tissues. Purified nematocysts are loaded with a drug and induced to discharge into the patient's skin, thus, executing sub dermal drug delivery. Currently, in order to initiate the process of nematocyst isolation from cnidarian tissue, specimens must be manually induced to extrude endogenous, nematocyst rich threads, which are subsequently cut with a dissecting scissors and siphoned into the first of several nematocyst isolation media. Cnidarian stinging cells have never been produced commercially but have been isolated for research purposes in order to study the protein toxins they contain and to decipher the capsule's structural properties. Yet, the great majority of the cnidome remains unexplored, due to the technical difficulties of isolating most stinging cells from surrounding tissues.

Martin, R. Management of nematocysts in the alimentary tract and cnidosacs of the aeolid nudibranch gastropod Cratena peregrina, Mar. Biol. 143, 533-541, 2003, describes feeding experiments which track the fate of nematocysts as they pass through the alimentary canal of an aeolid nudibranch gastropod, Cratena peregrine, to the digestive gland in the dorsal appendages, the cerata, to the cnidosacs, and also in the faeces. Masses of exposed, undigested and structurally intact nematocysts were discarded in the faeces. After release, in contact with seawater, cnidosac nematocysts were able to discharge. It is concluded that a large proportion of the nematocysts ingested with the food are not digested, but are eliminated, structurally and functionally intact, via the tips of the cerata and structurally intact via the alimentary canal.

One of the most common problems affecting captive reef systems is the proliferation of biofouling sea-anemones (Anthozoa: Actinaria) of the genus Aiptasia. These clonal, zooxanthellate sea anemones often dominate marine fouling communities causing economic damage to unprotected, submerged infrastructure. In marine aquaria, these anemone “pests” compete with coral colonies for space and light, ultimately causing the demise of captive corals through competitive interactions. They also cause physical damage to other valuable organisms (e.g., fish), and esthetic damage to ornamentally pleasing aquaria. Alternative methods of alleviating captive reef systems of these pests exist, yet ultimately, the problem persists.

### SUMMARY OF THE INVENTION

One object of the present invention is to provide a method for propagating benthic marine invertebrates including distinct life cycle stages thereof in an artificial environment.

Another object of the present invention is to provide an apparatus for propagating benthic marine invertebrates.

A further object of the invention is to provide applications utilizing propagated Molluscs.

In one aspect of the invention, there is provided an apparatus for propagating benthic marine invertebrates in water comprising:
a. a culture cell;
b. a sedimentation chamber in fluid connection with the culture cell; and
c. a pump in fluid connection with the culture cell and with the sedimentation chamber capable of pumping water from the sedimentation chamber to the culture cell.

The culture cell is a container capable of containing water in which the benthic marine invertebrates grow and propagate. The cell has an inlet and an outlet to enable cycling of the water.

Any one or more of the following features or deigns and configurations may be incorporated in the device and utilized in the method according to the present invention:
In one embodiment, the inlet is located in the lower portion of the cell and the outlet is located in the upper portion of the cell so that the water flow is in an upward direction.
The outlets may be openings in the upper circumference of the culture cell, optimally covered by a mesh to prevent escape of the marine animals.
In another embodiment, removable sheets, e.g. nylon mesh or PVC, of e.g. 500 um, are fixed to the inner vertical walls of the culture cell for attachment thereto of the benthic marine invertebrates, e.g, sea anemones.
The culture cell is preferably positioned in the apparatus so that when the apparatus is placed on a level surface, the vertical axis of the cell is perpendicular to the surface.
The apparatus may contain a plurality of culture cells.
The sedimentation chamber is a container in which the temperature and quality of the culture cell water is controlled. The sedimentation chamber is in fluid connection with the culture cell and, generally, in close proximity to it. In one embodiment, the culture cell is encompassed by the sedimentation chamber.
The sedimentation chamber may have one or more inlets, a feed outlet connected to the inlet of the culture cell, and a waste outlet at its bottom end for draining particulate waste.
The upper end of the sedimentation chamber may be open or closed. Preferably, the bottom end of the sedimentation chamber has a conical shape.
The pump may be any conventional fluid pump. In one embodiment, the pump is located in the sedimentation chamber. Optimally, a heating element is associated with the pump for controlled heating of the water. The ambient temperature is conducive to the propagation of benthic marine invertebrates and there is no need to control the temperature of the water. In many cases, however, the water temperature must be controlled. Therefore, in one embodiment, the apparatus includes a heat exchange tank which encompasses the culture cell and sedimentation chamber.
According to another embodiment, the culture cell is located within the sedimentation chamber which in turn is located within the heat exchange tank.
The heat exchange tank may contain a fluid such as water and may be in thermal or fluid connection with a thermostat-regulated heater and/or a thermostat-regulated cooler to control the fluid temperature.
In one embodiment, a thermostat-regulated heater is located in the sedimentation chamber.
By one example, the outer wall of the heat exchange tank is temperature insulated while the inner wall bordering the sedimentation chamber and/or culture cell is not insulated.
The vertical walls of the culture cell and/or sedimentation chamber and/or heat exchange tank are made of an opaque material. The purpose of this feature of the invention is to mimic the light conditions in the sea where the light source is almost exclusively from above, i.e. substrates to which marine animals attach are generally opaque. Thus, in a further embodiment, the apparatus comprises a light source which is typically positioned above the culture cell.

The water flow in the apparatus may be as follows. Water (e.g. filtered seawater) is pumped into the sedimentation chamber, through an inlet or through the open upper end, and from there into the culture cell by the pump. The water in the culture cell flows back into the sedimentation chamber. Waste water exits the sedimentation chamber through the waste outlet. A stand pipe may be attached to the waste outlet so as to control the water level in the sedimentation chamber by raising or lowering it. This is called an open circuit. An alternate configuration is a closed circuit in which the waste water is recycled back into the sedimentation chamber after being passed through a filter, such as a biofilter water system. Planktonic nudibranch larvae tend to get caught by surface tension and die. The upward water flow in the culture cell eliminates surface tension, vastly improving culture survival percentages.

The desired flow regime is determined by the type of invertebrate to be cultured, so that this regime is as close as possible to the natural conditions, to thereby obtain laminar flow or turbulent flow or vortex.

In one embodiment, an array of apparatuses may be constructed, having a common sedimentation chamber and/or heat exchange tank. Optionally a common pump is used.

According to another aspect of the invention, there is provided a method for propagating benthic marine invertebrates comprising culturing adult benthic marine invertebrates in a non-fouled environment containing biofouled egg laying substrates. In an alternate aspect of the invention, there is provided a method for propagating benthic marine invertebrates comprising culturing the invertebrates in a water environment in an apparatus according to the invention.

In the present specification, benthic marine invertebrates are invertebrate animals which dwell on a solid surface (substrate) in a water body. These organisms generally inhabit or are physically connected to submerged solid substrates in aqueous environments e.g., coral reefs, rocky shores, sand beds, artificial substrates. The invention also relates to the propagation of non-benthic stages in the life cycle of benthic marine invertebrates (Molluscs and Cnidarians), such as planktonic nudibranch larvae (veliger larvae) and planktonic Cnidarian larvae (planulae). This may be carried out by controlling the flow speed (lower than the larval swimming speed) in the culture cell and constraining the outlet size of the culture cell (smaller than the larvae's minimal dimension), or by lifting water from the sedimentation chamber and dripping it into the culture cell, creating a force that pulls the larvae down and out of the surface tension. In the later case, the culture cell may be constructed in toto of nylon mesh smaller than the larvae's minimal dimension.

According to one embodiment of this aspect of the invention, the benthic marine invertebrates are Cnidarians. In one embodiment the Cnidarians are Anthozoa. In another embodiment, the Anthozoa are Actinaria. In another embodiment, the Actinaria are acontiate sea anemones. In another embodiment, the sea anemone is Aiptasia diaphana.

In an alternate embodiment of this aspect of the invention, the benthic marine invertebrates are Molluscs. In one embodiment the Molluscs are Gastropoda. In another embodiment, the Gastropoda are Opisthobranchia. In another embodiment, the Opisthobranchia are Nudibranchia. In another embodiment, the Nudibranchia are Aeolids. In another embodiment, the Aeolid is Spurilla neapolitana.

In a further embodiment of this aspect of the invention, the molluscs are fed Cnidaria. In one embodiment, the Cnidaria are Anthozoa. In a further embodiment, the Anthozoa are Actinaria.

One example of this aspect of the invention is the propagation of Nudibranch animals. It has been discovered that Nudibranchs, and particularly S. neapolitana, will not lay egg bundles on clean, antiseptic surfaces. Furthermore, egg bundles should not be physically disturbed, i.e. removed from the substrate they are attached to, as developing embryos are extremely susceptible to shearing forces. In accordance with one embodiment, adult Nudibranchs are placed in a non-fouled environment (which is defined as an environment from which bio-organic residues have been removed) containing biofouled (defined as coated by a bio-organic film) egg laying substrates. The Nudibranchs preferably lay the egg bundles on the egg laying substrates. The egg bundles may then be transferred to a larval culture media by transferring them in toto on the egg laying substrates without applying shearing forces to them.

It has also been discovered that the temperature of the environment during embryonic development is crucial to the successful propagation of the animals. Thus, a constant, controlled temperature must be maintained during the various stages of Mollusc development.

One application of the invention is based on the understanding that by co-cultivation of Cnidarians and marine Molluscs (which live with and feed upon them) under suitable culturing media, it is possible to isolate and purify from the faeces of the marine Molluscs, intact, undischarged, dischargeable nematocysts, which may be readily activated, i.e. discharged, when placed in a standard discharge inducing medium. The isolated nematocysts may be used for research purposes, as well as for commercial production.

Thus, according to yet another aspect of the invention, there is provided a method for isolating Cnidarian nematocysts comprising isolating the nematocysts from Mollusc feces. In one embodiment, the Mollusc is propagated on Cnidarians, and the Mollusc's feces is collected.

The present invention provides un-discharged, dischargeable (i.e. functional), nematocysts isolated from Mollusc faeces. The nematocysts may be isolated, for example, by density gradient centrifugation. The media may be multi-density, i.e. two or more layers of ascending or descending densities, or mono-density, i.e. one layer of media at a specified density. Isolated nematocysts may be used in the nematocyst-mediated drug delivery industry as biological micro-syringes and also for the “milking” of nematocyst venoms. As such, the invention provides access to novel cnidarian venoms.

Another application of the invention relates to controlling the growth of unwanted sea anemones in an aquarium by introducing Nudibranches into the aquarium. Aeolid nudibranchs (Gastropoda) are generally considered specialist predators. The present invention shows, for example, that the nudibranch Spurilla neapolitana readily feeds on sea anemones (Cnidaria: Actinaria) e.g Aiptasia diaphana but avoids stony corals (Cnidaria: Scieractinia). These nudibranchs are extremely efficient in selectively alleviating ornamental aquaria of anemone pests.

### BRIEF DESCRIPTION OF THE DRAWINGS

In order to understand the invention and to see how it may be carried out in practice, embodiments will now be described, by way of non-limiting examples only, with reference to the accompanying drawings, in which:

FIG. 1 is top view of a three dimensional representation of an apparatus according to one embodiment of the invention;

FIG. 2 is a longitudinal sectional view of an closed-circuit configuration apparatus according to the invention;

FIG. 3 is a longitudinal sectional view of an open-circuit configuration apparatus according to the invention;

FIG. 4 illustrates an embodiment wherein the sedimentation tank is non-integrated with the heat exchange tank;

FIGS. 5A and 5B are enlargements of the portion marked III in FIG. 2, illustrating a section of two different flow regime control members of the lower portion of the culture cell; and

FIG. 6 is an array of culture cells according to an embodiment of the invention/

### DETAILED DESCRIPTION OF EXEMPLARY EMBODIMENTS

### Apparatus for Propagating Marine Molluscs

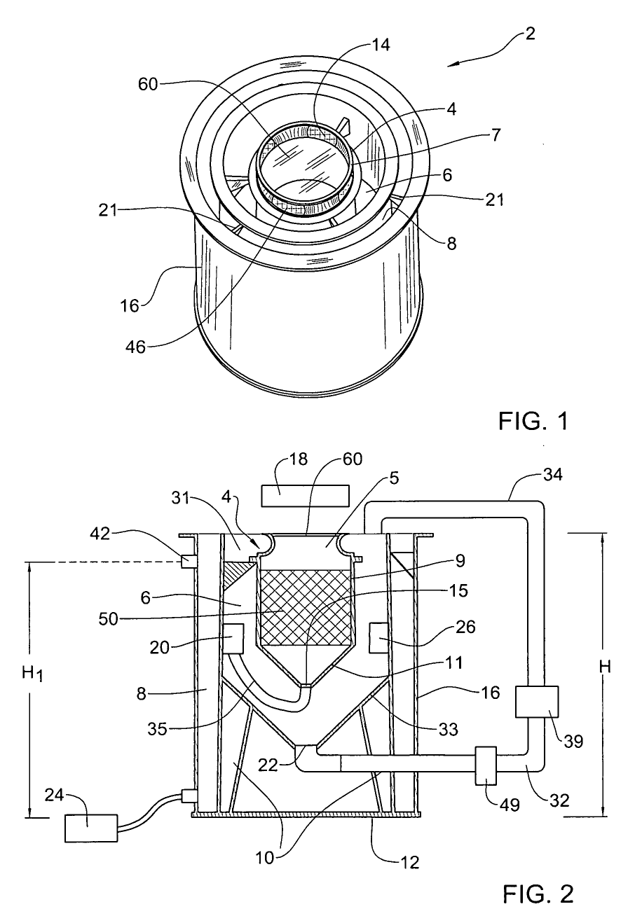

One embodiment of an apparatus in accordance with the invention will be described with reference to FIGS. 1 and 2. The apparatus 2 comprises a culture cell 4 placed on three radial supports 3 within a sedimentation chamber 6 which in turn is encompassed by a heat exchange tank 8. The culture cell 4, the sedimentation chamber 6 and the heat exchange tank 8 are concentric and have equi-leveled top edges extending at height H.

The culture cell 4 comprises an upper portion 5 with a top edge 7, an intermediate cylindrical portion 9 and a lower, intermediate cone-like shaped portion 11 fitted with an inlet port 15 at its lowermost end. The upper portion 5 has four spaced apart outlet ports 14 allowing fluid passage from the culture cell 4 to the sedimentation chamber 6. The outlet ports 14 may be covered by a mesh 46 to prevent escape of the marine animals.

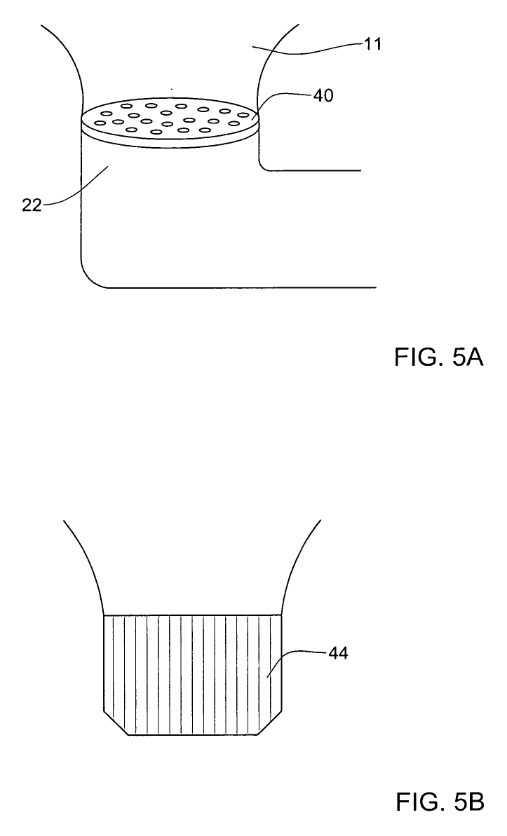

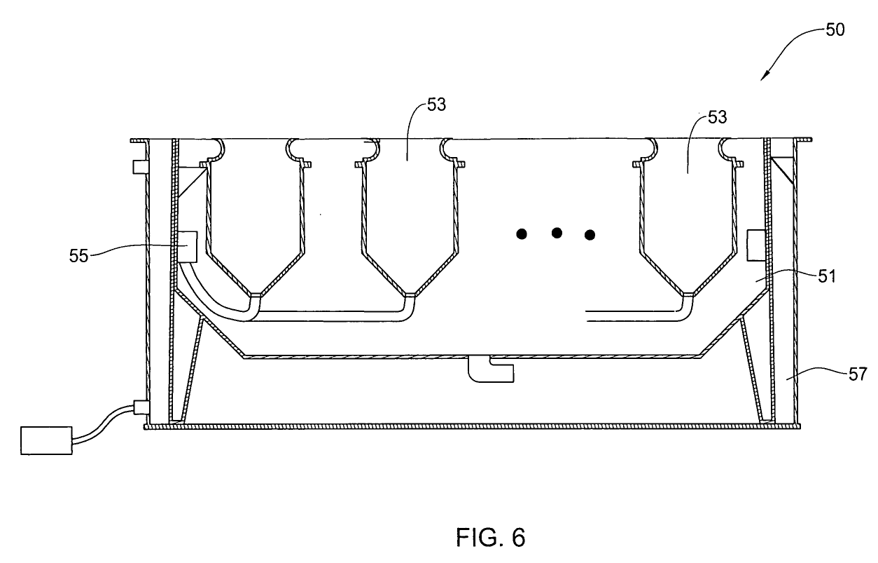

The culture cell 4 further comprises a perforated disc 40 (FIG. 5A) disposed at the inlet port 15, for control of the flow regime into the culture cell 4. Optionally, the inlet port 15 of the culture cell 4 comprises an arrangement such as a nozzle 44 (FIG. 5B) suited for generating a desired flow regime, as discussed hereinafter in further detail. The top edge 7 of the culture cell 4 may be covered by a transparent cover 60 so as to allow control of light wave length transfer therethrough and at the same time to prevent an air transfer. The cylindrical portion 9 of the culture cell 4 is fitted with a removable sheet 50 conforming the shape of the cylindrical portion 9 which may be used as a substrate for sessile, benthic invertebrates, e.g sea anemones, and may also be raised above the water outlet 14, thus containing larvae and small anemones. Optionally, the removable sheet may be coaxial cylindrical or helical.

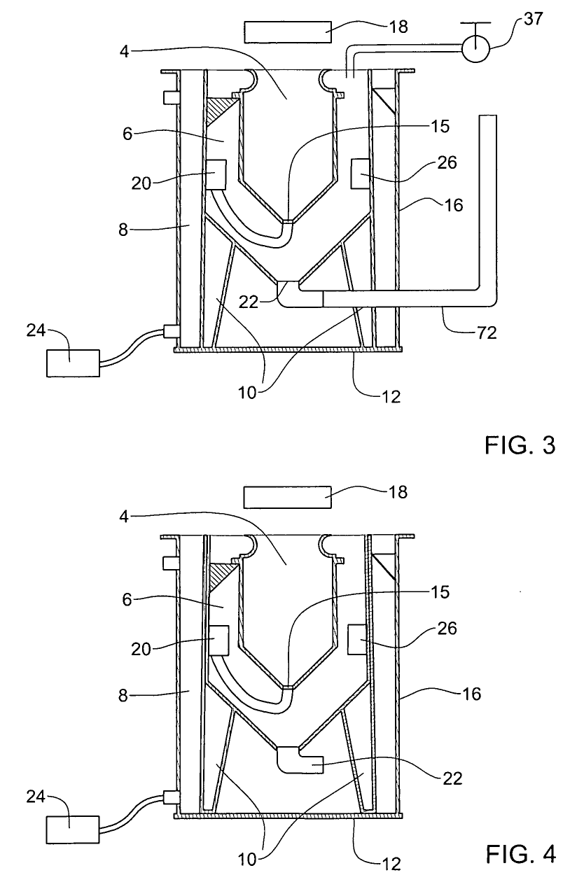

The sedimentation chamber 6 has a top edge 31, which may be open or closed, through which fresh filtered seawater is supplied from a water source 37 (FIG. 3), or circulated seawater via filter 39 and conduit 34 (FIG. 2). The sedimentation chamber 6 is formed with a conical lower portion 33 having a waste outlet 22 for discharging waste water through a conduit 32 (FIG. 2) or the conduit 72 (FIG. 3) attached thereto. The sedimentation chamber 6 is supported over support legs 10 so as to distance it from the floor 12 of the apparatus 2, and is supported by radial supports 21 extending from the heat exchange tank 8.

The sedimentation chamber 6 further comprises a pump 20 fixed to its wall and connected to the inlet port 15 of the culture cell 4 by means of a pipe 35 through which the water is supplied from the sedimentation chamber 6 to the culture cell 4. Together with the perforated disc 40 (FIG. 5A) the pump 20 controls the flow regime within the culture cell 4 by changing the flow rate of the water supplied to the culture cell 4.

The heat exchange 6 has an outlet 42 for prevention of overflow which otherwise could possibly flow into the sedimentation tank. Height H₁ of the outlet 42 is lower than height H.

The apparatus 2 has several configurations of operation. The first configuration is a “closed circuit” configuration (FIG. 2), in which once sea water is filled in the sedimentation chamber 6 there is water circulation between the sedimentation chamber 6 and the culture cell 4. In particular, the water is pumped from the sedimentation chamber 6 to the culture cell 4 by means of pump 20, and is discharged through the outlet ports 14 of the culture cell 4 and then back to the sedimentation chamber 6 where the water is recycled through conduit 32 by circulation pump 49, through the filter 39 (e.g. a bio-filter) before it is returned to the sedimentation chamber 6 through a conduit 34.

The “closed circuit” configuration, as illustrated in FIG. 2, is especially useful when there is no available access to the sea-water or when the products are to be used in medical or cosmetic applications, as such applications require relatively high quality of water purification.

The second configuration is an “open circuit” configuration, as illustrated in FIG. 3, in which fresh sea water is constantly added to the sedimentation chamber 6 though a self regulated dripper (e.g. at a water flow rate of 5 l/h, 8 l/h, 16 l/h), which by means of pump 20 is pumped into the culture cell 4, then discharged to the sedimentation chamber 6 through outlets 14, and partially removed through the waste outlet 22 of the sedimentation chamber 6, mimicking thereby the natural flushing of the water. In this configuration water level within the sedimentation chamber 6 is controlled by the U-shaped vessel 72 according to the ‘communicating vessels’ principle.

Due to the upward flow direction i.e. from the inlet 11 towards the outlets 14, there is no accumulation of the sediment in the culture cell and it is drained out of the sedimentation chamber through the waste outlet 22.

The “open circuit” configuration is useful when a substantially unlimited access to sea-water is available and when the main purpose of the apparatus is culturing the animals for ornamentation or agriculture purposes, rather than isolating their cells for other applications.

The apparatus according to the invention 2 may be further operated by a combination of the “open circuit” and “closed circuit” configurations, where the fresh water is occasionally added to the sedimentation chamber 6.

As mentioned above, the pump 20 together with the perforated disc 40 control the flow regime within the culture chamber. The desired flow regime is determined by the kind of invertebrates to cultured, so that this regime is as close as possible to the natural conditions. For example, flow within the laminar flow regime was obtained using the perforated disk 40 at flow rates smaller then 200 l/h and turbulent flow was obtained at flow rates exceeding about 600 l/h. Without the perforated disk turbulent flow was obtained at flow rates between about 100-600 l/h. flow rates above those specified will generate a vortex. In particular, when culturing Actinarias the optimal conditions require turbulent flow in the range of flow rates that prevent vortex creation.

Feed is occasionally added to the culture cell 4 through a feeding inlet (not shown), in which case it may be added manually or automatically. Alternatively, the feed may be added directly through the pipe 35, e.g. by a feed tube articulated thereto. The latter option may be particularly useful when the apparatus comprises more then one culture cell, as will be further explained.

Temperature is controlled and stabilized by a closed water system in the heat exchange tank 8, pumped through a water chiller 24, which is not necessarily in water connection with the culture cell 4. A thermostat regulated heater 26 is placed in the sedimentation chamber 6 and/or in the heat exchange tank 8 to further stabilize the temperature. Both the heat exchange tank 8 and the sedimentation chamber 6 may be covered to avoid loss of heat through evaporation. The outer wall 16 of the heat exchange tank 8 is made of an opaque, heat insulating material such as PVC.

A light source 18 is positioned above the culture cell 4, allowing continuous control of light intensity, light quality and the light/dark (L:D) cycle from a single, controlled source. For example, the light source 18 may be florescent lamps with spectral peaks conducive for photosynthesis such as ˜420 nm, ˜630 nm, ˜664 nm and ˜670 nm may be used at intensities in the range of 10-1000 μe, preferably about 200 μe, for controlling the light parameters.

FIG. 6 shows another example of an apparatus 50 according to the present invention. The apparatus 50 comprises an elongated sedimentation chamber 51, and a plurality of culture cells 53 received therewith. The culture cells 53, which may be similar to the culture cell 4 described above, are arranged in parallel and connected to one common pump 55 within the sedimentation chamber 51. The apparatus 50 may further comprise an elongated heat exchange tank 57 similar to the exchange tank 8 described above.

The apparatus according to the present invention may comprise a plurality of plates arranged in parallel replacing the culture cells within the sedimentation chamber.

According to one configuration illustrated in FIG. 4 the sedimentation chamber 6 with the associated culture cell 4 is a stand-alone unit suitable for use without the heat exchange tank 8. this unit is useful in particular with the open circuit configuration where a continues sea water supply is provided at sea temperature, thus making any heat regulation redundant.

According to this configuration the support legs 10 are filled with a heavy substance (e.g. sand, gravel, etc.) so that when the device is used in conjunction with a heat exchange chamber it may be introduced therein and it will then sink into the position of FIG. 4. Alternatively, other fastening options may be used e.g. bayonet coupling, bolts or other mechanical fasteners.

### Cultivation and Culture of Marine Nudibranchs

It has been found that embryonic development rates are positively correlated to temperature. Development is disturbed when oviposition occurs at temperatures above 26° C., and egg bundles may untimely disintegrate causing the “abortion” of unhatched eggs. The most effective temperature for broodstock culture and embryonic development in egg bundle culture is within the range of 15-26° C., more preferably 18-25° C., most preferably 24° C.

It has been found that S. neapolitana will not lay egg bundles on clean, aseptic surfaces. In order to culture nudibranch larvae, an aseptic culture media is needed. Furthermore, egg bundles may not be physically disturbed as developing embryos are extremely susceptible. In addition, Nudibranchs are nocturnal, being cryptic when exposed to light. Therefore, an egg laying substrate consisting of halved 40 mm PVC pipes may be used to provide a shelter from light. Nudibranch aquaria are kept aseptic while a biofilm is allowed to accumulate upon the halved pipes. Eggs are laid on the pipes alone. These are transferred without disturbing the egg bundle, into the egg culture media and kept at 24° C. until hatching, whereupon larvae are removed and transferred into larval culture media.

The larvae may be cultured at a temperature in the range of 15-30° C., preferably 25° C. in an aseptic growth cell as described above. Flow speed is set at less than 0.2 cm s⁻¹, preferably 0.05-0.2 cm s⁻¹ the swimming speed of the larvae. Alternatively, water may be dripped from above, using an airlift system. A 25-75 μm nylon mesh, preferably 50 □m, retains the larvae within the cell. Alternatively, the cell may be constructed of nylon mesh.

Larval diet: Larvae are planktotrophic. The preferred diet consists of 10⁵ Isochrysis galbana cells/ml+10³ Tetraselmis tetrathele cells/ml.

Culture media: Larvae are susceptible to pathogens. They are preferably cultured in 50 ug/ml streptomycin and 60 ug/ml penicillin.

Metamorphosis induction: Larvae reach competency at age 25-30 days post oviposition. Larvae are induced to settle and metamorphose using A. diaphana solutes.

Post larval culture: 48 h post metamorphosis induction, post larvae begin preying on sea anemones. Sea anemones are preferably introduced into the culture medium 48 h post induction.

### Nematocyst Isolation

Starved nudibranchs are placed in a cylindrical cell with a conical outlet at its bottom. The cell contains anemone coated PVC sheets. Fecal pellets collect at the bottom of the cell and are collected into a hyperosmotic nematocysts retention media by opening a valve at the bottom of the cell. Thus, it inhibits nematocyst discharge (as suggested by Blanquet R., Ionic effects on discharge of the isolated and in-situ nematocysts of the sea anemone Aiptasia pallida: a possible role of calcium. Comp. Biochem. Physiol., vol. 35, pp. 451-461, 1970). The faeces are then centrifuged on a Percol cushion (as modified from Marchini B., De Nuccio L., Mazzei M., Mariottini G. L., A fast centrifuge method for nematocysts isolation from Pelagia noctiluca Forskal (Cnidaria: Scyphozoa), Revisita di Biologia/Biology Forum, 97, 505-516, 2004; Domart-Coulon, I. J., Elbert, D. C., Scully, E. P., Calimlim P., S., Ostrander G. K. Arogonite crystallization in primary cell cultures of multicellular isolates from a hard coral, Pocillopora damicornis, PNAS, vol. 98, pp. 11885-11890, 2001). A gradient centrifugation (10%, 20%, up to 70% Percol) is used in order to effectively purify each type of nematocyst present in the nudibranch faeces.

Nematocysts purified by this procedure are induced to discharge by placing them in a hypo-osmotic media. Nematocyst activity is easily verified by monitoring their discharge under a light microscope.

1. An apparatus for propagating benthic marine invertebrates in water, comprising:
a culture cell;
a sedimentation chamber in fluid connection with the culture cell; and
a pump in fluid connection with the culture cell and with the sedimentation chamber capable of pumping water from the sedimentation chamber to the culture cell.

2. The apparatus according to claim 1, wherein the culture cell is fitted with one or more removable invertebrates beds received therein.

3. The apparatus according to claim 1, further comprising a heat exchange tank encompassing the culture cell and the sedimentation chamber.

4. The apparatus according to claim 2, wherein the removable invertebrates bed is a mesh.

5. The apparatus according to claim 1, further comprising a light source for illuminating the culture cell.

6. The apparatus according to claim 5, wherein the light source is positioned above the culture cell.

7. The apparatus of claim 1, further comprising a thermostat-regulated heater and/or a thermostat-regulated cooler.

8. The apparatus according to claim 1, wherein the water is pumped between the culture cell and sedimentation chamber in a closed circuit, wherein water removed from the sedimentation chamber is filtered and pumped back into the culture cell.

9. The apparatus according to claim 1, wherein the water is pumped between the culture cell and sedimentation chamber in an open circuit, wherein fresh sea water is continuously filled into the sedimentation chamber and is then pumped into the culture cell, with overflow water removed through a communicating vessel pipe.

10. The apparatus according to claim 2, wherein water is pumped into the culture cell from its bottom end in an upwards direction such that water flow flushes the invertebrates bed.

11. The apparatus according to claim 1, wherein flow regime into the culture cell is selected so as to mimic flow conditions corresponding with marine natural conditions of the invertebrates.

12. The apparatus according to claim 11, wherein the flow regime is governed by a flow regulator fitted at an inlet fluid inlet of the culture cell.

13. The apparatus according to claim 12, wherein the flow regulator is a perforated disc or a nozzle.

14. The apparatus according to claim 1, wherein overflow from the culture cell into the sedimentation chamber takes place through outlets at an upper end of the culture cell.

15. The apparatus according to claim 1, wherein the culture cell has a tapering bottom end, with a water inlet formed at lowermost portion thereof.

16. The apparatus according to claim 1, wherein the sedimentation chamber has a tapering bottom end, with a waste outlet formed at lowermost portion thereof.

17. The apparatus according to claim 1, comprising a plurality of culture cells, all being in flow communication with the sedimentation chamber.

18. The apparatus according to claim 3, wherein the sedimentation chamber is detachably received within the heat exchange tank.

19. The apparatus according to claim 1, wherein the benthic marine invertebrates are Molluscs.

20. A method for propagating benthic marine invertebrates comprising culturing the invertebrates in a water environment in an apparatus comprising a culture cell, a sedimentation chamber in fluid connection with the culture cell, and a pump in fluid connection with the culture cell and with the sedimentation chamber capable of pumping water from the sedimentation chamber to the culture cell.

21. The method according to claim 20, wherein the culture cell is fitted with one or more removable invertebrates beds received therein.

22. The method according to claim 21, wherein sea water is pumped into the culture cell from its bottom end in an upwards direction such that water flow flushes the invertebrates bed.

23. The method according to claim 20, wherein flow regime into the culture cell is selected so as to mimic flow conditions corresponding with marine natural conditions of the invertebrates.

24. The method according to claim 20, wherein overflow from the culture cell into the sedimentation chamber takes place through outlets at an upper end of the culture cell.

25. The method according to claim 20, wherein the culture cell has a tapering bottom end, with a water inlet formed at lowermost portion thereof.

26. The method according to claim 20, wherein the sedimentation chamber has a tapering bottom end, with a waste outlet formed at lowermost portion thereof.

27. The method according to claim 20, wherein the benthic marine invertebrates are Molluscs.

28. The method according to claim 27, wherein the Molluscs are fed Cnidaria.

29. A method according to claim 27, wherein egg bundles of the Molluscs are laid on an egg laying substrate covered by a bio organic film.

30. The method according to claim 18, wherein the water environment during embryo development is maintained at a constant temperature.

31. A method for isolating Cnidarian nematocysts comprising isolating the nematocysts from Mollusc feces.

32. The method according to claim 27, wherein the Mollusc is propagated on Cnidarians, and the Mollusc's feces is collected.

33. The method according to claim 31, wherein the nematocysts are isolated from the Molluscs' feces by centrifugation of the feces in a density gradient media.

34. The method according to claim 33, wherein the density gradient media is selected from the group consisting of Percol, Ficoll and sucrose.

35. The method according to claim 28 wherein the Molluscs are propagated according to the method comprising culturing the Molluscs in a water environment in an apparatus comprising a culture cell, a sedimentation chamber in fluid connection with the culture cell, and a pump in fluid connection with the culture cell and with the sedimentation chamber capable of pumping water from the sedimentation chamber to the culture cell.

36. The method according to claim 31, wherein Cnidarian nematocysts are obtained by propagating Molluscs comprising culturing the Molluscs in a water environment in an apparatus comprising a culture cell, a sedimentation chamber in fluid connection with the culture cell, and a pump in fluid connection with the culture cell and with the sedimentation chamber capable of pumping water from the sedimentation chamber to the culture cell.

37. A method for propagating benthic marine invertebrates comprising culturing adult benthic marine invertebrates in a non-fouled environment containing biofouled egg laying substrates.

38. A method for controlling the growth of unwanted sea anemones in an aquarium comprising introducing Nudibranches into the aquarium.
